(19) United States
(12) Patent Application Publication
(10) Pub. No.: US 2024/0307581 A1
(43) Pub. Date: Sep. 19, 2024

(54) AIR TREATMENT DEVICE

(71) Applicant: DAIKIN INDUSTRIES, LTD., Osaka-shi (JP)

(72) Inventors: Takayoshi OKAMOTO, Osaka-shi (JP); Tetsuya YAMASHITA, Osaka-shi (JP); Atsuo NAKASHIMA, Osaka-shi (JP); Masato MIYAGAMI, Osaka-shi (JP)

(21) Appl. No.: 18/676,290

(22) Filed: May 28, 2024

Related U.S. Application Data
(63) Continuation of application No. PCT/JP2022/038234, filed on Oct. 13, 2022.

(30) Foreign Application Priority Data
Nov. 29, 2021 (JP) 2021-193551

(51) Int. Cl. A61L 9/20

(52) U.S. Cl. CPC A61L 9/20; A61L 2209/134; A61L 2209/14

(57) ABSTRACT

An air treatment device includes a casing having an opening, an air passage formed in the casing, a first unit disposed in the air passage, a predetermined functional component disposed in the air passage, an irradiator configured to irradiate the functional component with ultraviolet rays, and a light blocking member arranged for air in the air passage to pass through the light blocking member. The first unit is removable from the casing through the opening. The light blocking member is arranged to block the ultraviolet rays emitted from the irradiator. The light blocking member is disposed between the first unit and the irradiator.

### CROSS-REFERENCE TO RELATED APPLICATIONS

This is a continuation of International Application No. PCT/JP2022/038234 filed on Oct. 13, 2022, which claims priority to Japanese Patent Application No. 2021-193551, filed on Nov. 29, 2021. The entire disclosures of these applications are incorporated by reference herein.

### BACKGROUND

### Technical Field

The present disclosure relates to an air treatment device.

### Background Art

An air purifier described in Japanese Unexamined Patent Publication No. 2015-143603 includes an air purifying filter and a humidifying unit. This air purifier includes a casing with an opening through which the humidifying unit is to be attached to and to be detached from the air purifier. The opening is provided with a removable door. A user may remove the door so that the opening is opened, and so the user can take a functional component such as a humidifying unit out of the casing.

### SUMMARY

A first aspect of the present disclosure is to an air treatment device including a casing having an opening, an air passage formed in the casing, a first unit disposed in the air passage, a predetermined functional component disposed in the air passage, an irradiator configured to irradiate the functional component with ultraviolet rays, and a light blocking member arranged for air in the air passage to pass therethrough. The first unit is removable from the casing through the opening. The light blocking member is arranged to block the ultraviolet rays emitted from the irradiator. The light blocking member is disposed between the first unit and the irradiator.

### BRIEF DESCRIPTION OF THE DRAWINGS

FIG. 1 is a perspective view illustrating an external appearance of an air treatment device according to an embodiment.

FIG. 2 is a perspective view of the cross section taken along line II-II shown in FIG. 1.

FIG. 3 is an exploded perspective view illustrating configurations of a fan unit and an air purification unit.

FIG. 4 illustrates a cross section of a humidifying chamber taken along line IV-IV shown in FIG. 1.

FIG. 5 is a block diagram showing the relationship between a control unit and various devices.

FIG. 6A, FIG. 6B and FIG. 6C illustrate perspective views of an air purifier showing an operation for opening a door member. FIG. 6A illustrates that the door member is closed. FIG. 6B illustrates that the door member is in the process of opening. FIG. 6C illustrates that the door member has been removed.

FIG. 7 is a plan view of a light blocking member.

FIG. 8 illustrates a cross section of an air purification chamber taken along line VIII-VIII shown in FIG. 1.

FIG. 9A is a diagram for describing reflections of ultraviolet rays having reached a first grille portion.

FIG. 9B is a diagram for describing reflections of ultraviolet rays having reached a second grille portion.

FIG. 10 is a block diagram showing the relationship between a control unit and various devices of an air treatment device according to a first variation.

FIG. 11 is a flowchart showing a control flow for an irradiator.

FIG. 12 is a refrigerant piping system diagram of an air treatment device according to a second variation.

FIG. 13 is a cross-sectional view illustrating a configuration of an air treatment device.

### DETAILED DESCRIPTION OF EMBODIMENT(S)

An embodiment of the present disclosure will be described with reference to the drawings. The following embodiment is merely an exemplary one in nature, and is not intended to limit the scope, applications, or use of the present invention. Features of the embodiment, variations, and other examples described below can be combined or partially substituted within the range where the present invention can be embodied.

### (1) General Configuration of Air Treatment Device

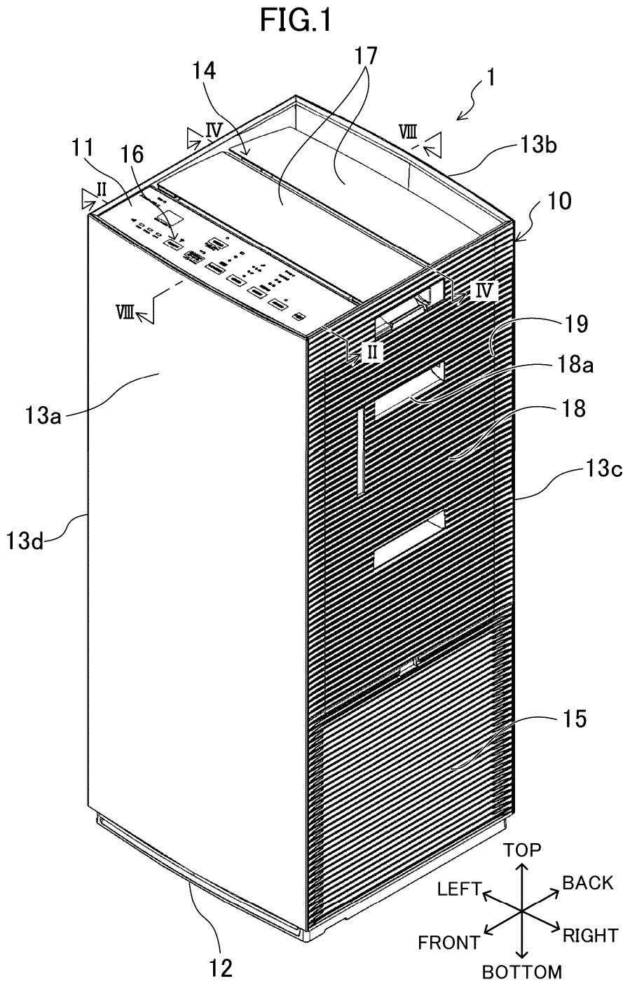

As illustrated in FIGS. 1 to 3, an air treatment device (1) of this embodiment is an air purifier (1) configured to purify air in an indoor space. The air purifier (1) is of a floor-standing type to be placed indoors. The air purifier (1) has a humidifying function of humidifying the air in the indoor space, in addition to an air purification function. A user may select the air purification function only. When the user selects the humidifying function, the air purification function is accompanied.

The air purifier (1) mainly includes a casing (10), a fan unit (U1), an air purification unit (U2), and a humidifying unit (U3). Each component will be described in detail.

### (2) Casing

The casing (10) is in a vertically long box shape. In the casing (10), an air blow chamber (2), an air purification chamber (3), and a humidifying chamber (4) are arranged from the bottom to the top. In the casing (10), an air passage (A) is formed. The air passage (A) enables communication between inlets (15) and outlets (14), which will be described later. The air in the air passage (A) flows through the fan unit (U1), the air purification unit (U2), and the humidifying unit (U3) in this order.

The casing (10) includes a top panel (11), a bottom plate (12), and four side plates (13). The top panel (11) is provided with an operation panel (16). The operation panel (16) includes a power switch for selecting the start or stop of the air purifier (1), a selection button for selecting the start or stop of the humidifying function, and the like. The operation panel (16) includes a display (not shown) for displaying information such as the indoor humidity and the room temperature at the present time. In the following description, “front”, “back”, “right”, and “left” indicate the directions when the operation panel (16) is viewed from the front.

The top panel (11) includes the outlets (14). The outlets (14) allow the air in the air passage (A) to be blown into the indoor space. The outlets (14) form another end of the air passage (A). The outlets (14) are located behind the operation panel (16). The outlets (14) extend over the entire transverse area. The outlets (14) are provided with two flaps (17). The flap (17) is in a horizontally long rectangular shape. The two flaps (17) are aligned in the front-to-back direction. The flap (17) is turnably supported by the top panel (11), and opens and closes the outlets (14) and adjusts the wind direction.

The four side plates (13) include a front panel (13a), a back panel (13b), a right side panel (13c), and a left side panel (13d). The right side panel (13c) and the left side panel (13d) each have a lower portion provided with the inlet (15). The inlets (15) form one end of the air passage (A). The inlets (15) allow the air in the indoor space to be sucked in. Specifically, the inlet (15) is provided below the center of each side panel (13c, 13d) and substantially over the entire transverse area (in the front-to-back direction) of each side panel (13c, 13d). The inlet (15) is provided with a prefilter (not shown). The prefilter is disposed to cover the inlet (15). This enables collection of relatively large dust and the like. The bottom plate (12) closes the lower ends of the side plates (13).

### (2-1) Opening

The casing (10) has an opening (19). The opening (19) is in a rectangular shape. The opening (19) is formed in a portion of the right side panel (13c) above the center of the right side panel (13c). The opening (19) is formed substantially over the entire transverse area (in the front-to-back direction) of the right side panel (13c). The opening (19) is provided with a door member (18). The door member (18) covers the opening (19).

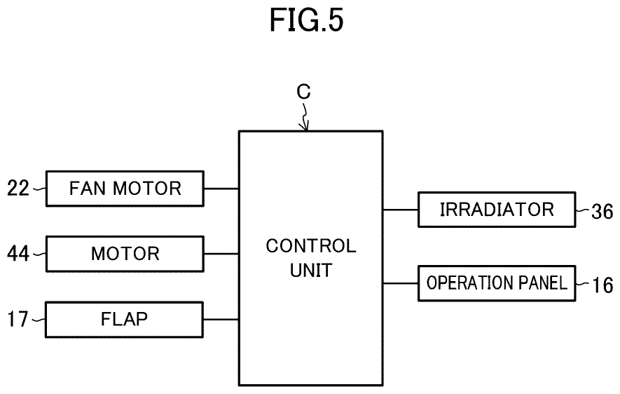

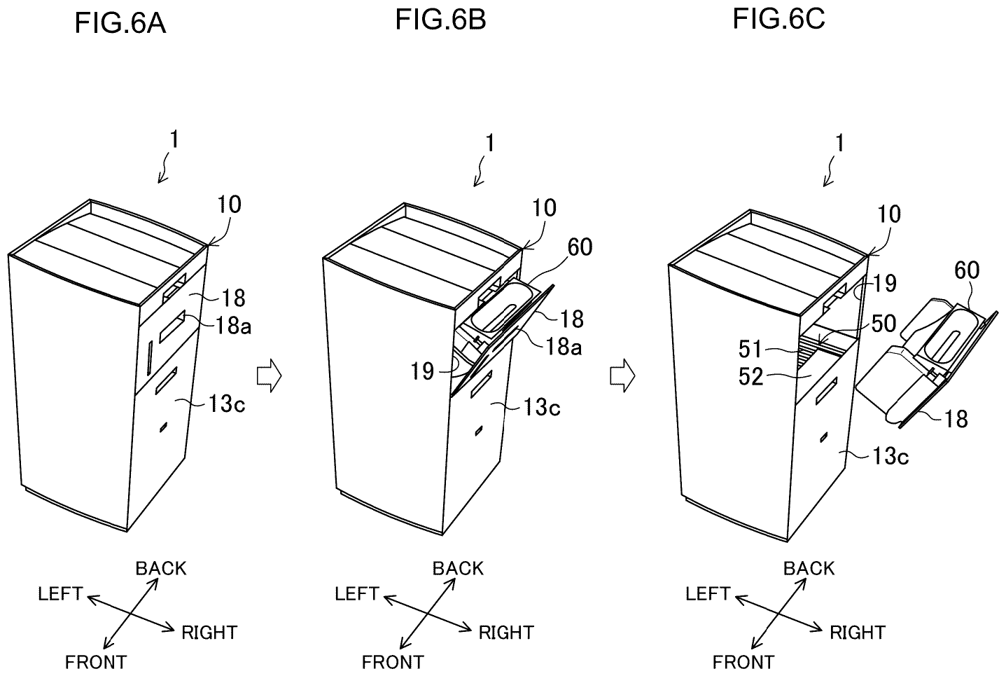

The door member (18) forms part of the right side panel (13c). The door member (18) can open and close the opening (19). Specifically, the lower end of the door member (18) is turnably supported by the casing (10). When a handle (18a) for the door member (18) is pulled toward the user (rightward), the panel surface of the door member (18) is inclined around the lower end of the door member (18) toward the user. Accordingly, the opening (19) is gradually opened. When the door member (18) is removed from the opening (19), the opening (19) is fully opened (see FIG. 6C).

### (3) Fan Unit

The fan unit (U1) is disposed in the air blow chamber (2). The fan unit (U1) faces the inlets (15). The fan unit (U1) includes a fan housing (23), a fan (21), and a fan motor (22).

In the fan housing (23), the fan (21) is disposed. The fan housing (23) has inflow ports (23a) and an outflow port (23b). The inflow ports (23a) are provided on the left and right sides of the fan housing (23). The inflow ports (23a) face the inlets (15) in the right-to-left direction. The inflow ports (23a) communicate with the inlets (15). The outflow port (23b) is provided on the upper surface of the fan housing (23). The outflow port (23b) communicates with the inside of the air purification unit (U2) to be described later. The inflow ports (23a) and the outflow port (23b) are formed in the fan housing (23). In this way, the internal space of the fan housing (23) forms part of the air passage (A).

The fan (21) transfers the air in the air passage (A) from the inlets (15) to the outlet (14). The fan (21) is a sirocco fan. The fan (21) is driven by the fan motor (22) so as to rotate. The fan (21) includes a plurality of blades (21a). The plurality of blades (21a) are arranged in a cylindrical shape around the drive shaft of the fan motor (22). The plurality of blades (21a) arranged in a cylindrical shape face the inflow ports (23a). As the fan motor (22) rotates, the plurality of blades (21a) rotate. With the rotation of the plurality of blades (21a), indoor air is taken into the fan housing (23) through the inlets (15) and the inflow ports (23a). The air in the fan housing (23) is transferred from the inflow ports (23a) to the outflow port (23b).

The air volume of the fan (21) is variable. Specifically, the number of rotations of the fan motor (22) is variable. The number of rotations of the fan motor (22) is switchable among a plurality of stages. The number of rotations of the fan motor (22) is controlled by a control unit (C).

### (4) Air Purification Unit

The air purification unit (U2) is disposed in the air purification chamber (3). The air purification unit (U2) includes a container (33), an irradiator (36), a primary filter (31), a secondary filter (32), and a light blocking member (50).

### (4-1) Container

The container (33) is a box-shaped member with an open top. The container (33) is made of a resin material. The container (33) contains the irradiator (36), the primary filter (31), the secondary filter (32), and the light blocking member (50). Specifically, in the container (33), the irradiator (36), the primary filter (31), the secondary filter (32), and the light blocking member (50) are arranged in this order from the bottom to the top.

The container (33) has a bottom plate portion (34) and a wall portion (35). The bottom plate portion (34) is substantially rectangular. The bottom plate portion (34) includes a pedestal (34a) and a bottom opening (34b). The pedestal (34a) is a portion on which the irradiator (36) is disposed. Specifically, the pedestal (34a) is located on the left side with respect to the center of the bottom plate portion (34). The upper surface of the pedestal (34a) is inclined upward from the right to the left. The bottom opening (34b) is rectangular. The bottom opening (34b) is located on the right side with respect to the center of the bottom plate portion (34). The bottom opening (34b) is connected to the outflow port (23b) of the fan housing (23). Thus, the air in the fan housing (23) flows into the container (33) through the outflow port (23b). The air in the container (33) flows out upward through the light blocking member (50). In this way, the internal space of the container (33) forms part of the air passage (A).

The wall portion (35) extends upward from the outer peripheral edge of the bottom plate portion (34). The wall portion (35) has an inner peripheral surface with a step (35a). The step (35a) has an annular shape. The step (35a) is closer to the upper end of the wall portion (35) than to the midpoint between the upper end and the lower end of the wall portion (35).

### (4-2) Irradiator

The irradiator (36) irradiates the primary filter (31) with ultraviolet rays. Specifically, the irradiator (36) includes a UV light source (36a) configured to emit ultraviolet rays. The irradiator (36) is disposed on the pedestal (34a). The irradiator (36) emits ultraviolet rays upward. The pedestal (34a) is located on the left side with respect to the center of the bottom plate portion (34), and has an upper surface inclined upward from the right to the left. Thus, the optical axis of the UV light source (36a) is tilted toward the center of the lower surface of the primary filter (31).

### (4-3) Primary Filter and Secondary Filter

The primary filter (31) and the secondary filter (32) are arranged so as to overlap each other in the vertical direction. Specifically, the top of the step (35a) is in contact with the lower surface of the primary filter (31), and the secondary filter (32) is placed above the primary filter (31). A gap is formed between the lower surface of the primary filter (31) and the upper surface of the bottom plate portion (34).

The primary filter (31) is an example of a functional component of the present disclosure. The primary filter (31) catches contaminants in the air. The primary filter (31) catches particles with a particle size of 10 μm to 50 μm, for example. The primary filter (31) is disposed in the air passage (A).

The secondary filter (32) is a filter having a pleated structure, and collects particles with a particle size of 0.7 μm. For example, a high efficiency particulate air (HEPA) filter that has a particle collection efficiency of 99.97% or more for particles with a particle size of 0.3 μm, or a medium/high-efficiency filter that collects particles with a particle size of 0.4 μm to 0.7 μm may be used.

### (4-4) Light Blocking Member

The light blocking member (50) is disposed to close the opening in the upper surface of the container (33). The light blocking member (50) is disposed above the primary filter (31) and the secondary filter (32). The light blocking member (50) is configured to allow the air in the air passage (A) to pass therethrough and to block the ultraviolet rays emitted from the irradiator (36). The light blocking member (50) is disposed at substantially the same height as the lower end of the opening (19). The light blocking member (50) can be removed from the container (33). The structure of the light blocking member (50) will be described in detail later.

### (5) Humidifying Unit

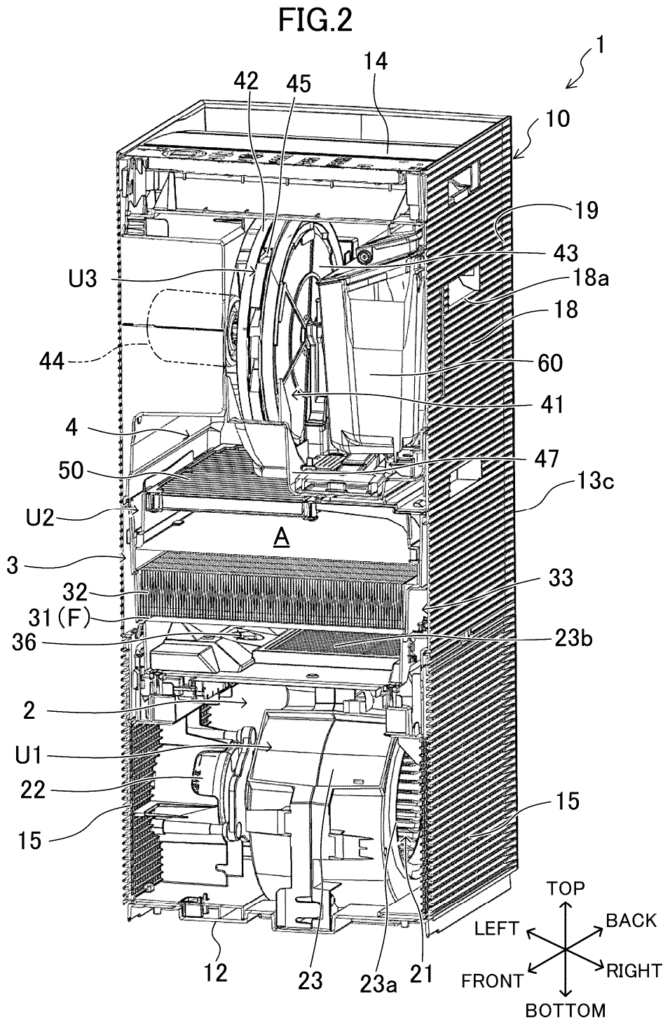

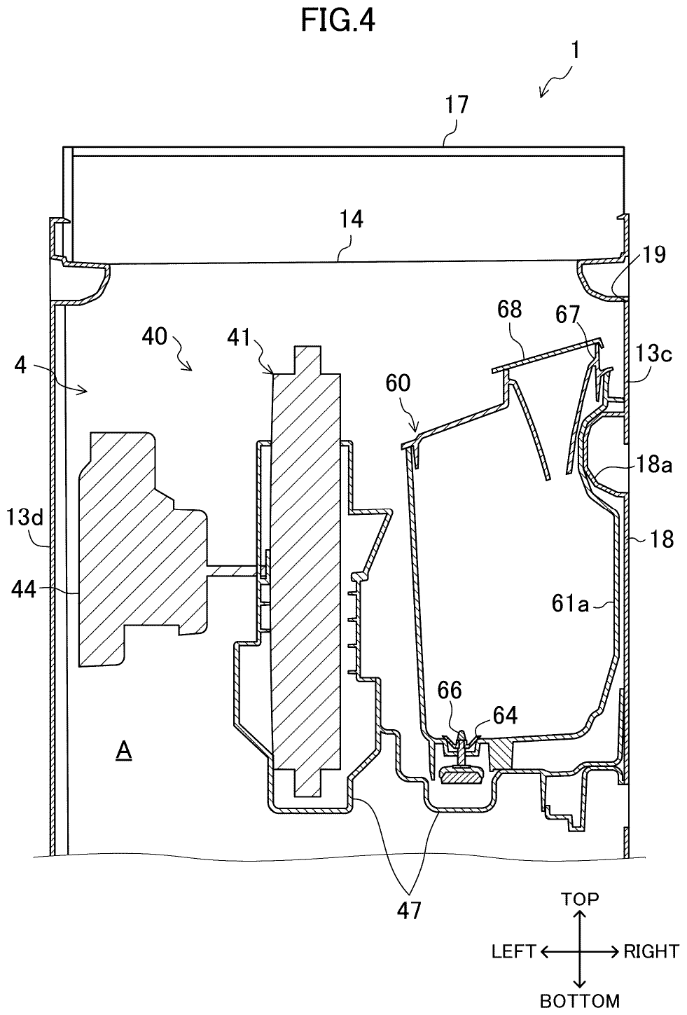

As illustrated in FIGS. 2 and 4, the humidifying unit (U3) is disposed in the humidifying chamber (4). The humidifying unit (U3) humidifies the air in the air passage (A). In the humidifying chamber (4), air flows from the left to the right. The humidifying unit (U3) is an example of a first unit (U3) of the present disclosure. The air purification unit (U2) and the humidifying unit (U3) are arranged in this order from the inlets (15) to the outlets (14) of the air passage (A). Specifically, the irradiator (36), the primary filter (31), the light blocking member (50), and the humidifying unit (U3) are arranged in this order from the one end to the other end of the air passage (A). The humidifying chamber (4) faces the opening (19). The humidifying unit (U3) is disposed in a right portion of the humidifying chamber (4). The humidifying unit (U3) is disposed near the opening (19). The humidifying unit (U3) includes a vaporization filter (41), a tray (47), and a tank (60).

### (5-1) Vaporization Filter

The vaporization filter (41) includes a frame (42) having an annular shape, and a disk-shaped vaporization material (43) fixed inside the frame (42). The vaporization filter (41) is positioned so that the air in the humidifying chamber (4) passes through the vaporization material (43). Specifically, the vaporization filter (41) is positioned so that the panel surface of the vaporization material (43) faces in the right-to-left direction. The center of the vaporization filter (41) is connected to the drive shaft of the motor (44). The motor (44) is disposed in a left portion of the humidifying chamber (4). When the motor (44) is energized, the drive shaft rotates. As the drive shaft of the motor (44) rotates, the vaporization filter (41) rotates.

The frame (42) has a lower end immersed in water stored in the tray (47). The frame (42) has an outer peripheral edge provided with a plurality of water scoops (45). With rotation of the vaporization filter (41), the water scoops (45) scoop up water in the tray (47) and supply the scooped water to the vaporization material (43).

The vaporization material (43) is made of a nonwoven fabric. The vaporization material (43) is fixed inside the frame (42). The vaporization material (43) retains the water supplied from the water scoops (45).

### (5-2) Tray

The tray (47) is disposed below the vaporization filter (41) and the tank (60). The tray (47) receives water stored in the tank (60). The tray (47) can store water to a height so that the lower end of the vaporization filter (41) is immersed in the water.

### (5-3) Tank

The tank (60) is disposed on the right of the vaporization filter (41). The tank (60) is fixed to the inner surface of the door member (18). The tank (60) has an upper surface provided with a water inlet (67). Water is supplied from the outside into the tank (60) through the water inlet (67). The tank (60) has a lower surface provided with a communication hole (64). The communication hole (64) allows the inside of the tank (60) to communicate with the tray (47). The communication hole (64) is provided with a water supply valve (66). When the water supply valve (66) is open, water in the tank (60) flows into the tray (47) through the communication hole (64). When the water supply valve (66) is closed, the water in the tank (60) does not flow into the tray (47).

### (6) Control Unit

As illustrated in FIG. 5, the air purifier (1) includes the control unit (C). The control unit (C) controls operations of the irradiator (36) and the air purifier (1). The control unit (C) includes a microcomputer and a memory device for storing software for operating the microcomputer.

The control unit (C) is connected to various devices of the air purifier (1) in a wired or wireless manner. For example, the control unit (C) is connected to the irradiator (36), the fan motor (22), the motor (44), and the operation panel (16).

The control unit (C) controls the operations of various devices based on predetermined operation commands (e.g., commands to start and stop the air purifier (1), commands to start and stop a humidifying operation, and commands to start and stop a drying operation) transmitted from the operation panel (16).

The control unit (C) switches the irradiator (36) between on and off states. The control unit (C) of this embodiment can turn the irradiator (36) on even with the humidifying unit (U3) removed from the casing (10). In other words, in the air purifier (1) of this embodiment, the irradiator (36) emits ultraviolet rays even with the humidifying unit (U3) removed from the casing (10). The control unit (C) may turn the irradiator (36) off at the end of operation of the air purifier (1), or may turn the irradiator (36) off when a predetermined time has elapsed since the end of operation of the air purifier (1).

### (7) Removal of Humidifying Unit

The humidifying unit (U3) is configured to be removable from the casing (10) through the opening (19). The details will be described below.

As illustrated in FIG. 6A, FIG. 6B and FIG. 6C, when the handle (18a) of the door member (18) is pulled toward the user (rightward), the opening (19) is made open (FIG. 6A and FIG. 6B). When the door member (18) is removed from the casing (10), the opening (19) is made completely open (FIG. 6B and FIG. 6C). At this time, the tank (60) of the humidifying unit (U3) is also removed from the casing (10) together with the door member (18).

Since the vaporization filter (41) and the tray (47) of the humidifying unit (U3) face the opening (19), the user puts his/her hand into the opening (19) to remove the vaporization filter (41) and the tray (47) from the casing (10). At this time, the humidifying unit (U3) is disposed near the opening (19). Thus, the user can remove the vaporization filter (41) and the tray (47) from the casing (10) without putting his/her hand deep inside (to the left end of) the casing (10).

The air purification unit (U2) of the air purifier (1) of this embodiment can be removed from the casing (10). Specifically, when the humidifying unit (U3) is removed from the casing (10), the humidifying chamber (4) has a space to which the air purification unit (U2) is movable. After putting his/her hand into the opening (19) and lifting the air purification unit (U2) to the same height as the opening (19), the user can remove the air purification unit (U2) from the casing (10) through the opening (19).

### (8) Operation of Air Purifier

Next, various operations of the air purifier (1) of this embodiment will be described.

### (8-1) Humidifying Operation

The humidifying operation includes purifying and humidifying sucked indoor air, and blowing the air into the indoor space again. When the humidifying operation is selected on the operation panel (16), the control unit (C) drives the fan motor (22) and the motor (44). The control unit (C) controls the number of rotations of the fan (21) and the vaporization filter (41) based on the set degree of humidification and the set air volume.

With the rotation of the fan (21), the indoor air having been sucked from the inlets (15) flows from the inflow ports (23a) into the fan housing (23). The air in the fan housing (23) is transferred from the outflow port (23b) into the container (33) of the air purification unit (U2). In this way, the indoor air having been sucked from the inlets (15) is transferred from the air blow chamber (2) to the air purification chamber (3). The air in the container (33) passes through the primary filter (31) and the secondary filter (32) in this order, and then is transferred to the humidifying chamber (4) through the light blocking member (50). The air having been transferred to the air purification chamber (3) is purified by passing through the air purification unit (U2).

The air having flowed into the humidifying chamber (4) is humidified by the humidifying unit (U3). Specifically, with the rotation of the vaporization filter (41), the water scoops (45) scoop up water in the tray (47) and supply the water to the vaporization material (43). The air flowing through the humidifying chamber (4) is humidified while passing through the vaporization material (43) containing moisture. In this time, the moisture is taken away from the vaporization material (43). However, since the vaporization filter (41) rotates, the water scoops (45) having scooped up water from the tray (47) supplies the water again. In this way, in the humidifying operation, with the rotation of the vaporization filter (41), the vaporization material (43) always contains moisture. This enables continuous humidification of the air passing through the vaporization filter (41).

The air having been humidified in the humidifying chamber (4) is blown indoors from the outlets (14). When the indoor humidity reaches a target value, the control unit (C) stops the humidifying operation. Accordingly, the humidifying operation stops. In the humidifying operation of this embodiment, the following air purification operation is performed at the same time.

### (8-2) Air Purification Operation

The air purification operation is an operation for purifying sucked indoor air. When the air purification operation is selected on the operation panel (16), the control unit (C) drives the fan motor (22). When the fan motor (22) is driven, in the air passage (A), the indoor air having been sucked from the inlets (15) flows toward the outlets (14). The control unit (C) controls the number of rotations of the fan (21) based on the set air volume. When only the air purification operation is performed, the motor (44) is not energized and thus the vaporization filter (41) does not rotate. Thus, the air in the humidifying chamber (4) is not humidified.

As described above, with the rotation of the fan (21), the indoor air having been sucked from the inlets (15) flows into the fan housing (23) through the inflow ports (23a). The air in the fan housing (23) is transferred from through the outflow port (23b) into the container (33) of the air purification unit (U2). In this way, the indoor air having been sucked from the inlets (15) is transferred from the air blow chamber (2) to the air purification chamber (3). The air in the container (33) passes through the primary filter (31) and the secondary filter (32) in this order, and then is transferred to the humidifying chamber (4) through the light blocking member (50). The air having been transferred to the air purification chamber (3) is purified by passing through the air purification unit (U2).

The air having flowed into the humidifying chamber (4) passes through the vaporization filter (41). When no humidifying operation is performed, the vaporization filter (41) stops. Thus, the air passing through the vaporization filter (41) is not humidified and is blown from the outlets (14) into the indoor space.

### (8-3) Drying Operation

The drying operation is an operation for drying the vaporization filter (41) by the air blown by the fan (21). According to the drying operation the vaporization filter (41) can be dried in a shorter time than according to natural drying, and thus the vaporization filter (41) can have anti-mold or anti-odor properties.

When the drying operation is selected on the operation panel (16), the control unit (C) drives the fan motor (22) to execute the drying operation. In the drying operation, the control unit (C) controls the number of rotations of the fan (21) to obtain the minimum air volume.

With the rotation of the fan (21), the indoor air having been sucked from the inlets (15) is transferred from the air blow chamber (2) to the air purification chamber (3) and then to the humidifying chamber (4) in this order. The air having flowed into the humidifying chamber (4) passes through the stopping vaporization filter (41). This air dries the vaporization material (43) of the vaporization filter (41). The air having passed through the vaporization filter (41) is blown into the room. After elapse of a predetermined time, the control unit (C) stops the drying operation.

### (9) Problems by Irradiator of Air Purification Unit

In the air purifier (1) including the air purification unit (U2) as described in this embodiment, the air having been sucked from the inlets (15) first passes through the primary filter (31). At this time, relatively large quantities of contaminants in the air are deposited on the primary filter (31). Thus, the irradiator (36) is disposed below the primary filter (31) to irradiate the lower surface of the primary filter (31) with ultraviolet rays. However, part of the ultraviolet rays emitted upward from below the primary filter (31) penetrate the primary filter (31) and the secondary filter (32), and further travel to a space above the air purification unit (U2). Thus, when the user puts his/her hand into the opening (19) to take the humidifying unit (U3) out of the opening (19) in the side surface of the casing (10), the user's hand is exposed to ultraviolet rays from the irradiator (36). Thus, the safety of the user cannot be ensured.

Thus, in the air purifier (1) of this embodiment, the light blocking member (50) is provided on the upper surface of the air purification unit (U2) to block part of the ultraviolet rays emitted upward from the irradiator (36) which penetrate the primary filter (31) and the secondary filter (32). The structure of the light blocking member (50) of this embodiment will be described in detail below.

### (10) Structure of Light Blocking Member

In this embodiment, the structure of the light blocking member (50) blocks part of ultraviolet rays. The light blocking member (50) of this embodiment is a member enabling ultraviolet rays emitted from the irradiator (36) toward the filters (31, 32) to less leak to the humidifying chamber (4).

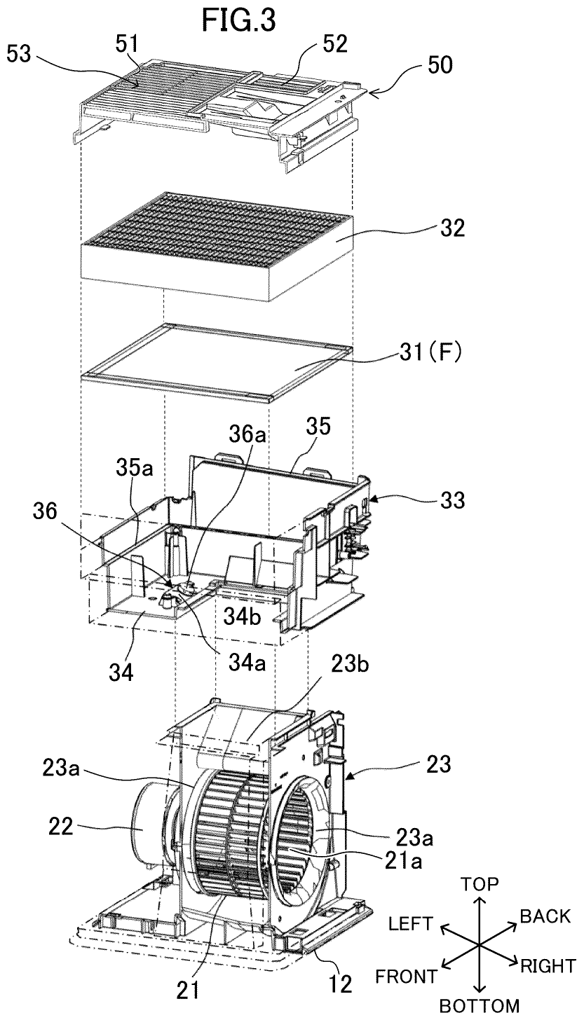

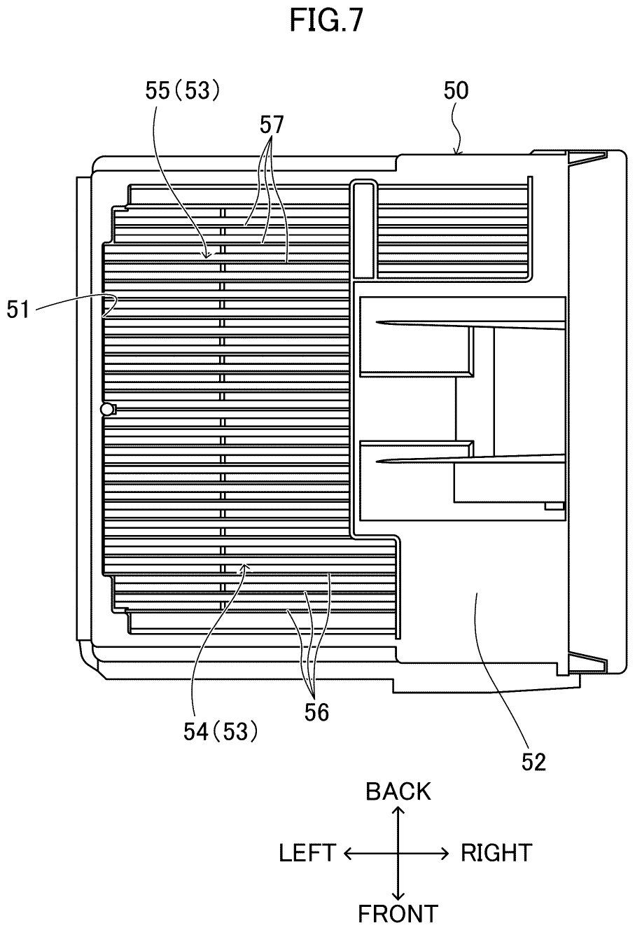

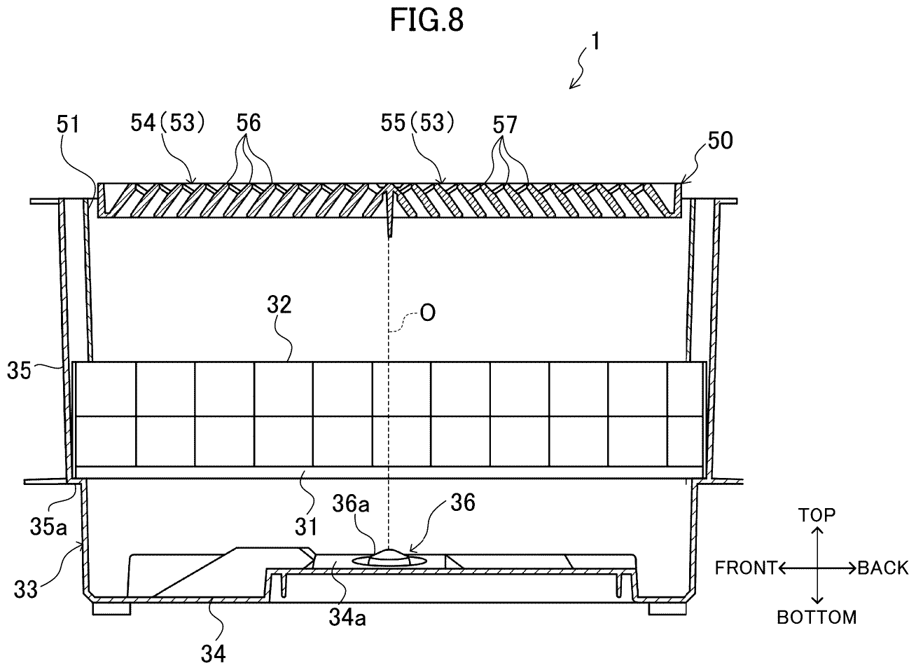

As illustrated in FIGS. 3, 7, and 8, the light blocking member (50) is in the shape of a box that opens downward. The light blocking member (50) is made of a resin material. The upper surface of the light blocking member (50) has a ventilation opening (51) and a flat portion (52). The ventilation opening (51) is a region through which the air in the air passage (A) passes. The ventilation opening (51) is an example of a first region (51) of the present disclosure. The ventilation opening (51) is formed in a left half region and a part of a right half region of the light blocking member (50) as viewed in a direction perpendicular to the paper plane of FIG. 7 (from above). The flat portion (52) extends leftward from the right end of the light blocking member (50). Specifically, the flat portion (52) is located in a substantially right half portion of the light blocking member (50) as viewed in the direction perpendicular to the paper plane of FIG. 7 (from above). A region where the flat portion (52) is formed as described above does not allow the air in the air passage (A) to pass therethrough. The region where the flat portion (52) is formed is a second region of the present disclosure. The region where the flat portion (52) is formed is closer to the opening (19) of the right side panel (13c) than the ventilation opening (51) (see FIG. 6C) is.

The light blocking member (50) includes a grille (53). The grille (53) is configured as a resin member. The grille (53) is disposed below the upper surface of the light blocking member (50). The grille (53) includes a first grille portion (54) and a second grille portion (55).

The first grille portion (54) is disposed in a front half region of the upper surface of the light blocking member (50) with respect to the center of the upper surface in the front-to-back direction. The first grille portion (54) includes a plurality of first plate members (56). The first plate members (56) are long and slender so as to extend between the right end and the left end of the light blocking member (50). The plurality of first plate members (56) are spaced apart by a predetermined distance from one another in the front-to-back direction, and are arranged parallel to one another. The first grille portion (54) is exposed from the ventilation opening (51) as viewed in the direction perpendicular to the paper plane of FIG. 7 (from above).

Each first plate member (56) is inclined backward and obliquely upward from the lower side to the upper side thereof. In other words, each first plate member (56) is inclined with respect to the upper surface of the light blocking member (50) so that the upper end of the first plate member (56) is located backward of the lower end thereof. In this way, each first plate member (56) is inclined with respect to the optical axis O of the irradiator (36) in a cross-sectional view taken along the plane orthogonal to the longitudinal direction of the first plate member (56).

The second grille portion (55) occupies a back half region of the upper surface of the light blocking member (50) with respect to the center of the upper surface in the front-to-back direction. The second grille portion (55) includes a plurality of second plate members (57). The second plate members (57) are long and slender so as to extend between the right end and the left end of the light blocking member (50). The plurality of second plate members (57) are spaced apart by a predetermined distance from one another in the front-to-back direction, and are arranged parallel to one another. The second grille portion (55) is exposed from the ventilation opening (51) as viewed in the direction perpendicular to the paper plane of FIG. 7 (from above).

Each second plate member (57) is inclined forward and obliquely upward from the lower side to the upper side thereof. In other words, each second plate member (57) is inclined with respect to the upper surface of the light blocking member (50) so that the upper end of the second plate member (57) is located forward of the lower end thereof. In this way, each second plate member (57) is inclined with respect to the optical axis O of the irradiator (36) in a cross-sectional view taken along the plane orthogonal to the longitudinal direction of the second plate member (57).

The first plate members (56) are symmetric to the second plate members (57) with respect to the optical axis O of the irradiator (36) in a cross-sectional view taken along the plane orthogonal to the longitudinal direction of the plate members (56, 57). The upper end of the first plate member (56) at the back end of the first grille portion (54) is in contact with the upper end of the second plate member (57) at the front end of the second grille portion (55).

The irradiator (36) is disposed midway between the front end and the back end of the light blocking member (50) when the light blocking member (50) is viewed from directly above. In other words, the irradiator (36) is disposed between the first plate member (56) and the second plate member (57) adjacent to each other in the front-to-back direction. Ultraviolet rays having passed from the irradiator (36) through the primary filter (31) and the secondary filter (32) and traveling forward of the location of the UV light source (36a) reach the first grille portion (54). Ultraviolet rays traveling backward of the location of the irradiator (36) reach the second grille portion (55). Ultraviolet rays traveling directly upward from the irradiator (36) reach a region between the first plate member (56) at the back end of the first grille portion (54) and the second plate member (57) at the front end of the second grille portion (55).

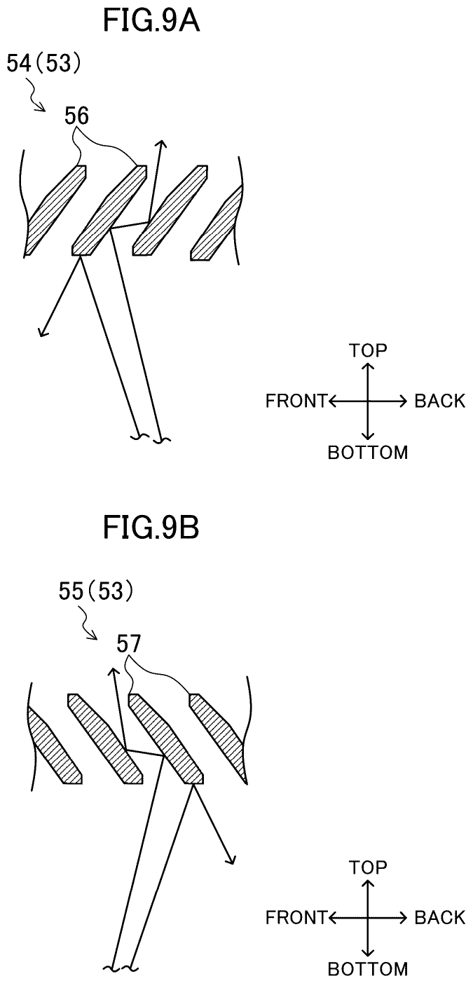

As indicated by the arrows in FIG. 9A, the ultraviolet ray reaching the first grille portion (54) is reflected by the lower surface of the first plate member (56). Specifically, the ultraviolet ray incident on the lower surface of the first plate member (56) is reflected downward, and thus do not thus pass through the light blocking member (50). After being reflected by the lower surface of the first plate members (56), part of the ultraviolet rays travels toward and is further reflected by the upper surface of the adjacent first plate member (56). In this way, the ultraviolet ray is attenuated by being reflected a plurality of times between the first plate members (56) adjacent to each other. Accordingly, even if this ultraviolet ray passes through the light blocking member (50), the human body is less affected.

As indicated by the arrows in FIG. 9B, the ultraviolet ray having reached the second grille portion (55) is also reflected by the lower surface of the second plate member (57), and thus does not pass through the light blocking member (50). After being reflected by the lower surface of the second plate member (57), part of the ultraviolet rays travels toward and is further reflected by the upper surface of the adjacent second plate member (57). In this way, the ultraviolet ray is attenuated by being reflected a plurality of times between the second plate members (57) adjacent to each other. Accordingly, even if the ultraviolet ray passes through the light blocking member (50), the human body is less affected.

### (11) Features

(11-1)

The air purifier (1) of this embodiment includes the casing (10) having the opening (19); the humidifying unit (U3) (first unit) removable from the casing (10) through the opening (19); the irradiator (36) configured to irradiate the primary filter (31) (functional component (F)) with ultraviolet rays; and the light blocking member (50) configured to block the ultraviolet rays emitted from the irradiator (36). The light blocking member (50) is disposed between the humidifying unit (U3) and the irradiator (36).

According to this embodiment, the ultraviolet rays emitted toward the primary filter (31) are blocked by the light blocking member (50), and thus the amount of ultraviolet rays reaching the humidifying unit (U3) can be reduced. Thus, when the user puts his/her hand into the opening (19) to take the humidifying unit (U3) out of the casing (10), the user's hand can be less exposed to ultraviolet rays. Thus, the safety of the user can be ensured.

(11-2)

In the air purifier (1) of this embodiment, the irradiator (36), the primary filter (31), the light blocking member (50), and the humidifying unit (U3) are arranged in this order from the one end to the other end of the air passage (A). In this way, ultraviolet rays emitted from the irradiator (36) travel through the primary filter (31), the light blocking member (50), and the humidifying unit (U3) in this order, whereas the light blocking member (50) can reduce the amount of ultraviolet rays reaching the humidifying unit (U3).

(11-3)

The air purifier (1) of this embodiment includes the container (33) disposed in the air passage (A) and containing the irradiator (36), the primary filter (31), and the light blocking member (50). In this way, the irradiator (36), the functional component (F), and the light blocking member (50) can be contained as a unit in the container (33). Thus, the irradiator (36), the functional component (F), and the light blocking member (50) are detachable together.

(11-4)

In the air purifier (1) of this embodiment, the light blocking member (50) includes the ventilation opening (51) (first region) through which the air in the air passage (A) is allowed to pass, and the flat portion (52) (second region) through which the air in the air passage (A) is not allowed to pass. The flat portion (52) is closer to the opening (19) than the ventilation opening (51).

The flat portion (52) is less likely to allow the ultraviolet rays from the irradiator (36) to pass therethrough than the ventilation opening (51). Thus, the amount of ultraviolet rays in a space over the flat portion (52) is smaller than the amount of ultraviolet rays in a space over the ventilation opening (51). Thus, by the flat portion (52) being closer to the opening (19) than the ventilation opening (51), the safety of the user putting his/her hand into the opening (19) can be ensured.

(11-5)

In the air purifier (1) of this embodiment, the humidifying unit (U3) and the flat portion (52) are arranged so that they at least partially overlap each other when viewed in the direction of air flow in the air passage (A). In the humidifying chamber (4), a space on the left of the humidifying unit (U3) vertically communicate with the ventilation opening (51), and thus the air flow resistance of air flowing from the air purification unit (U2) into the humidifying unit (U3) can be reduced.

(11-6)

In the air purifier (1) of this embodiment, the primary filter (31) is a filter (31) configured to catch contaminants in the air. By emission of the ultraviolet rays from the irradiator (36), the contaminants deposited on the primary filter (31) can be removed. Accordingly, the primary filter (31) can be kept clean.

### (12) Variations

The foregoing embodiment may be modified as the following variations. Differences from the foregoing embodiment will be mainly described below.

### (12-1) First Variation

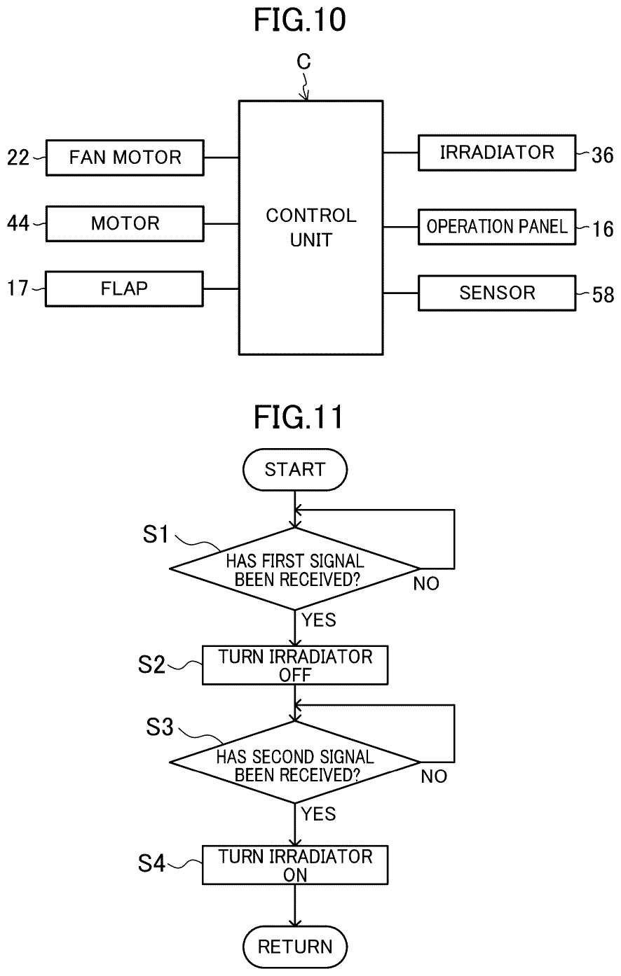

An air purifier (1) of a variation includes a light blocking member (50) configured to be detachable from the air passage (A). Specifically, the light blocking member (50) is configured to be detachable from the container (33). As shown in FIG. 10, the air purifier (1) of the variation includes a sensor (58) configured to sense that the light blocking member (50) has moved from the container (33). The control unit (C) switches the irradiator (36) between on and off states through a signal output from the sensor (58). For example, when, after the humidifying unit (U3) has been taken out of the casing (10), the user puts his/her hand into the opening (19) to remove the light blocking member (50) from the container (33), the sensor (58) senses that the light blocking member (50) has moved, and then outputs a predetermined signal. The control unit (C) that has received the signal from the sensor (58) turns the irradiator (36) off. A control flow for the control unit (C) will be specifically described with reference to FIG. 11.

In step S1, the control unit (C) determines whether or not a first signal has been received from the sensor (58). The first signal is a signal that is output when the light blocking member (50) is removed from the container (33). If the control unit (C) determines that the first signal has been received (YES in step S1), step S2 is executed. If the control unit (C) has not received the first signal (NO in step S1), step S1 is executed again.

In step S2, the control unit (C) turns the irradiator (36) off.

In step S3, the control unit (C) determines whether or not a second signal has been received from the sensor (58). The second signal is a signal that is output when the light blocking member (50) is installed in the container (33). If the control unit (C) determines that the second signal has been received (YES in step S3), step S4 is executed. If the control unit (C) has not received the second signal (NO in step S3), step S3 is executed again.

In step S4, the control unit (C) turns the irradiator (36) on.

As described above, in the air purifier (1) of the variation, the emission of ultraviolet rays from the irradiator (36) is stopped when the light blocking member (50) is to be removed. Thus, the safety of the user having put his/her hand into the opening (19) can be ensured.

### (12-2) Second Variation

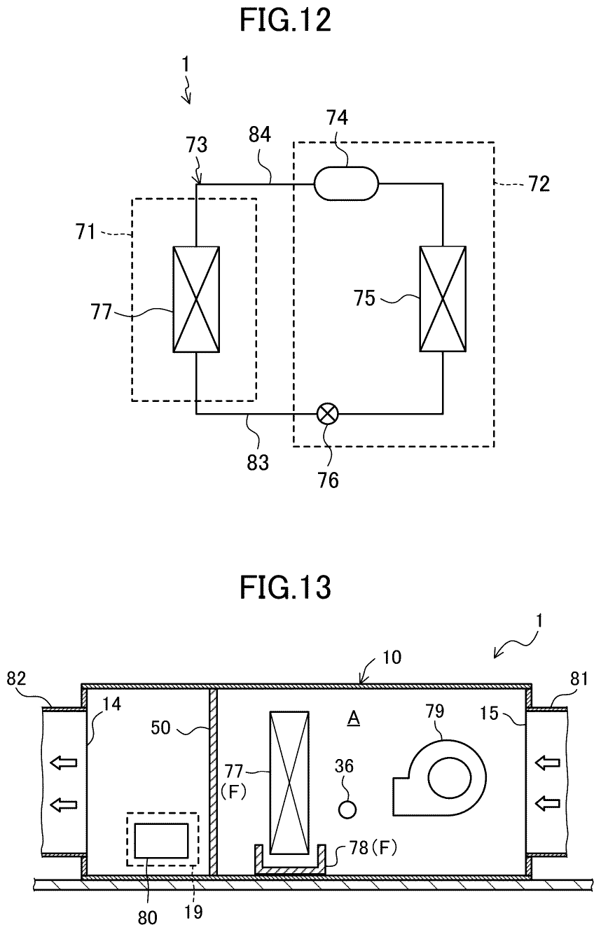

An air treatment device (1) of a second variation is an air conditioner (1). As shown in FIGS. 12 and 13, the air conditioner (1) includes an indoor unit (71) and an outdoor unit (72). In the air conditioner (1) of this variation, the indoor unit (71) and the outdoor unit (72) are connected together through a liquid connection pipe (83) and a gas connection pipe (84) to form a refrigerant circuit (73). The refrigerant circuit (73) is connected to a compressor (74), an outdoor heat exchanger (75), an expansion valve (76), and an indoor heat exchanger (77) in this order.

The outdoor unit (72) is placed outdoors. The outdoor unit (72) includes the compressor (74), the outdoor heat exchanger (75), and the expansion valve (76).

The compressor (74) sucks and compresses a low-pressure gas refrigerant. The compressor (74) discharges the compressed refrigerant.

The outdoor heat exchanger (75) functions as a condenser. The outdoor heat exchanger (75) exchanges heat between outdoor air and a refrigerant.

The expansion valve (76) decompresses the refrigerant. The expansion valve (76) merely needs to be connected to the liquid connection pipe (83) of the refrigerant circuit (73), and may be provided in the indoor unit (71).

The indoor unit (71) of this variation is a duct unit placed behind the ceiling. The indoor unit (71) includes a casing (10) having a horizontally elongated rectangular parallelepiped shape; an indoor fan (79); an irradiator (36); the indoor heat exchanger (77); a drain pan (78); a light blocking member (50); and an electric component unit (80).

The casing (10) has sidewalls facing each other in the longitudinal direction, one of which has an inlet (15) and the other one of which has an outlet (14). The inlet (15) is connected to a suction duct (81) communicating with an indoor space. The outlet (14) is connected to a blow-out duct (82) communicating with the indoor space. The internal space of the casing (10) includes an air passage (A) that allows the inlet (15) and the outlet (14) to communicate with each other.

The indoor fan (79) is disposed near the inlet (15) of the air passage (A). The indoor fan (79) is a sirocco fan. The indoor fan (79) transfers the air in the air passage (A) from the inlet (15) to the outlet (14).

The irradiator (36) of this variation is disposed downstream of the indoor fan (79) along the air flow in the air passage (A). The irradiator (36) emits ultraviolet rays toward the indoor heat exchanger (77) and the drain pan (78).

The indoor heat exchanger (77) is disposed downstream of the irradiator (36) along the air flow in the air passage (A). The indoor heat exchanger (77) functions as a condenser. The indoor heat exchanger (77) is a fin-and-tube heat exchanger. The indoor heat exchanger (77) exchanges heat between the air in the air passage (A) and the refrigerant. The indoor heat exchanger (77) is a functional component (F) of the present disclosure.

The drain pan (78) is disposed below the indoor heat exchanger (77). The drain pan (78) collects water generated from the indoor heat exchanger (77). The water stored in the drain pan (78) is discharged to the outside through a drain pipe (not shown). The drain pan (78) is a functional component (F) of the present disclosure.

In the air passage (A), the light blocking member (50) is disposed in a portion downstream of the drain pan (78) and the indoor heat exchanger (77) along the air flow and upstream of the location at which the opening (19) is formed along the air flow. The light blocking member (50) is supported by the inner peripheral surface of the casing (10). The flat portion (52) of the light blocking member (50) is closer to the opening (19) than the grille (53) is.

The electric component unit (80) is disposed downstream of the light blocking member (50) in the air passage (A). The electric component unit (80) is positioned so that the electric component unit (80) can be taken out of the casing (10) through the opening (19). The electric component unit (80) is disposed near the opening (19). The electric component unit (80) is a first unit (U3) of the present disclosure.

When the air conditioner (1) is operated, the indoor fan (79) causes indoor air to flow into the inlet (15) through the suction duct (81). The air that has flowed into the inlet (15) flows through the air passage (A) toward the outlet (14). The air in the air passage (A) is cooled by a heat exchange with the refrigerant in the indoor heat exchanger (77) functioning as an evaporator. The cooled air passes through the grille (53), flows into the blow-out duct (82) through the outlet (14), and is blown into the indoor space.

In the air passage (A), the air around the indoor heat exchanger (77) is cooled to a temperature equal to or lower than the dew point temperature. Accordingly, water droplets are generated on the indoor heat exchanger (77), and eventually fall into the drain pan (78).

The indoor heat exchanger (77) and the drain pan (78) are irradiated with ultraviolet rays by the irradiator (36). Thus, bacteria are less likely to appear in water deposited on the evaporator and the drain pan (78).

Part of ultraviolet rays travelling from the irradiator (36) toward the indoor heat exchanger (77) and the drain pan (78) reach the light blocking member (50). These ultraviolet rays can less leak upward of the light blocking member (50) by being blocked by the light blocking member (50). As a result, the safety of the user who is putting his/her hand into the opening (19) to take out the electric component unit (80) can be ensured.

### (13) Other Embodiments

The above-described embodiment may be modified as follows.

The control unit (C) of the air purifier (1) may turn the irradiator (36) off when the user puts his/her hand into the opening (19) to remove the humidifying unit (U3). Thus, the safety of the user can be ensured. For example, even if, due to a malfunction in the control unit (C), the irradiator (36) fails to be turned off when the humidifying unit (U3) is taken out, the light blocking member (50) of the air purifier (1) blocks ultraviolet rays emitted from the irradiator (36). Thus, the safety of the user can be ensured.

In the foregoing embodiment, the functional component (F) may include the primary filter (31) and the secondary filter (32), or may include the secondary filter (32) only.

The functional component (F) may be the humidifying unit (U3). In this case, the air purifier (1) is configured so that the light blocking member (50) is disposed above the humidifying unit (U3).

The air purifier (1) of the foregoing embodiment may include an electric dust collection unit, a deodorizing filter, a dehumidifying unit, and the like, as the first unit (U3).

While the embodiments and variations thereof have been described above, it will be understood that various changes in form and details may be made without departing from the spirit and scope of the claims. The foregoing embodiment and variation thereof may be combined and replaced with each other without deteriorating the intended functions of the present disclosure. The expressions of “first,” “second,” . . . described above are used to distinguish the terms to which these expressions are given, and do not limit the number and order of the terms.

As can be seen from the foregoing description, the present disclosure is useful for an air treatment device.

1. An air treatment device comprising:
a casing having an opening;
an air passage formed in the casing;
a first unit disposed in the air passage, the first unit being removable from the casing through the opening;
a predetermined functional component disposed in the air passage;
an irradiator configured to irradiate the functional component with ultraviolet rays; and
a light blocking member arranged for air in the air passage to pass therethrough, the light blocking member being arranged to block the ultraviolet rays emitted from the irradiator,
the light blocking member being disposed between the first unit and the irradiator.

2. The air treatment device of claim 1, wherein
the irradiator, the functional component, the light blocking member, and the first unit are arranged in order from one end to an other end of the air passage.

3. The air treatment device of claim 1, further comprising:
a container disposed in the air passage,
the container containing the irradiator, the functional component, and the light blocking member.

4. The air treatment device of claim 2, further comprising:
a container disposed in the air passage,
the container containing the irradiator, the functional component, and the light blocking member.

5. The air treatment device of claim 1, wherein
the light blocking member is configured to be detachable from the air passage, and
when the light blocking member is removed from the air passage, the irradiator stops emitting ultraviolet rays.

6. The air treatment device of claim 2, wherein
the light blocking member is configured to be detachable from the air passage, and
when the light blocking member is removed from the air passage, the irradiator stops emitting ultraviolet rays.

7. The air treatment device of claim 3, wherein
the light blocking member is configured to be detachable from the air passage, and
when the light blocking member is removed from the air passage, the irradiator stops emitting ultraviolet rays.

8. The air treatment device of claim 1, wherein
the light blocking member includes
a first region through which the air in the air passage is allowed to pass, and
a second region through which the air in the air passage is not allowed to pass, and
the second region is closer to the opening than the first region.

9. The air treatment device of claim 2, wherein
the light blocking member includes
a first region through which the air in the air passage is allowed to pass, and
a second region through which the air in the air passage is not allowed to pass, and
the second region is closer to the opening than the first region.

10. The air treatment device of claim 3, wherein
the light blocking member includes
a first region through which the air in the air passage is allowed to pass, and
a second region through which the air in the air passage is not allowed to pass, and
the second region is closer to the opening than the first region.

11. The air treatment device of claim 5, wherein
the light blocking member includes
a first region through which the air in the air passage is allowed to pass, and
a second region through which the air in the air passage is not allowed to pass, and
the second region is closer to the opening than the first region.

12. The air treatment device of claim 8, wherein
the first unit and the second region at least partially overlap each other when viewed along a direction of air flow in the air passage.

13. The air treatment device of claim 9, wherein
the first unit and the second region at least partially overlap each other when viewed along a direction of air flow in the air passage.

14. The air treatment device of claim 10, wherein
the first unit and the second region at least partially overlap each other when viewed along a direction of air flow in the air passage.

15. The air treatment device of claim 11, wherein
the first unit and the second region at least partially overlap each other when viewed along a direction of air flow in the air passage.

16. The air treatment device of claim 1, wherein
the functional component is a filter configured to catch a contaminant in air.
